(19) United States
(12) Patent Application Publication
(10) Pub. No.: US 2002/0013261 A1
(43) Pub. Date: Jan. 31, 2002

(54) Methods and compositions for promoting angiogenesis using polyethylene glycol (PEG) polymers

(76) Inventors: Robert Pawliuk, Medford, MA (US); Philippe Leboulch, Charlestown, MA (US)

Correspondence Address: LAHIVE & COCKFIELD, 28 STATE STREET, BOSTON, MA, 02109, US

(21) Appl. No.: 09/860,831

(22) Filed: May 18, 2001

Related U.S. Application Data
(60) Provisional application No. 60/205,346, filed on May 18, 2000.
(60) Provisional application No. 60/211,323, filed on Jun. 14, 2000.

(51) Int. Cl. A61K 48/00; A61K 38/18; A61K 31/715; A61K 31/075

(52) U.S. Cl. 514/2; 514/54; 514/44; 514/718

(57) ABSTRACT

Novel methods and compositions for stimulating angiogenesis, particularly at regions of myocardial and peripheral tissue ischemia are disclosed. Angiogenesis is promoted or enhanced by contacting a polyethylene glycol (PEG) polymer, such as a PEG mono-, di-, tri-, or tetraacrylate containing a photoinitator (eosin Y) and a radical generator (triethanolamine) and a reaction accelerator (n-vinyl pyrrolidine), with an area of tissue ischemia. The PEG polymer can be applied alone or in conjunction with angiogenic proteins or genes encoding angiogenic proteins.

### RELATED INFORMATION

This application claims priority to U.S. Provisional Patent Application Serial No. 60/205,346, filed on May 18, 2000, and to U.S. Provisional Patent Application Serial No. 60/211,323, filed on Jun. 14, 2000, both of which are incorporated by reference herein in their entirety. The contents of all patents, patent applications, and references cited throughout this specification also are hereby incorporated by reference in their entireties.

### BACKGROUND OF THE INVENTION

The depletion of oxygen supply to due to obstructed or inadequate blood supply is the common pathological state associated with various tissue ischemias, including myocardial ischemia, ischaemic bowel disease, and peripheral ischemia. The alleviation of tissue ischemia is critically dependent upon angiogenesis, the process by which new capillaries are generated from existing vasculature. The spontaneous growth of new blood vessels provide collateral circulation surrounding an occluded area, improves blood flow, and alleviates the symptoms caused by the ischemia. Although surgery or angioplasty may help to revascularize ischemic regions in some cases, the extent, complexity and location of the arterial lesions which cause the occlusion often prohibits such treatment.

Alternative methods for the treatment of chronic ischemia have focused on the direct injection of recombinant angiogenic proteins or expression vectors (both viral and non-viral) containing genes which encode angiogenic factors. Purified recombinant VEGF-A and basic fibroblast growth factor (bFGF) have been demonstrated to elicit a modest but significant vascularization following injection into ischemic skeletal muscle tissue in a rabbit model of chronic limb ischemia. In addition, direct injection of vectors containing cDNA encoding VEGF-A has also been shown to induce a modest stimulation of angiogenesis in ischemia animal models in both skeletal and cardiac muscle. However, all of these methods have significant limitations. The limited half-lives of many of the angiogenic proteins used in these approaches often necessitates repeated injections of large quantities of recombinant protein, thus rendering the technique impractical. Moreover, gene expression from vectors is often transient in nature since these vectors do not integrate efficiently into mammalian genomes. As a consequence, sustained expression of angiogenic factors from such vectors often drops precipitously in less than 2 weeks. This is considerably less than the 2-3 month treatment period required for optimal revascularization. Accordingly, the density and quality of new vasculature generated by these techniques is generally sub-optimal and insufficient to produce a sustained alleviation of ischemia.

Other related methods for the treatment of chronic ischemia have focused on the transplatation of autologous or non-autologous cells which have been genetically modified such that they produce angiogenic proteins. In one such approach, a subject's endogenous cells are isolated, cultured, and transfected with expression vectors encoding angiogenic proteins. Following in vitro manipulations, these cells are injected back into the patient at the site of tissue ischemia. The drawbacks of this approach include the time and effort required to isolate, culture and transfect target cells from each individual patient, as well as difficulties in securing sustained expression of angiogenic proteins. These disadvantages conspire with sub-optimal cell survival and differentiation states of the cells following injection to degrade the viability of this approach. In another approach, cells are obtained from a non-patient source, or even a non-human source, and manipulated in the manner described above. At least some of the problems associated with either of these approaches can be attributed to the patient's own immune system, which will try to remove such modified cells from the site of injection, particularly those which are from a non-patient source.

Accordingly, improved therapies for promoting tissue angiogenesis and for administering therapeutic proteins in a safe, effective and controlled manner to treat tissue ischemia are needed.

### BRIEF DESCRIPTION OF THE DRAWINGS

FIG. 1 is a schematic diagram outlining a particular PEG polymerization procedure.

FIG. 2 shows the angiogenic response following injection of PEG polymer capsules suspended in Matrigel. The angiogenic response to PEG capsules is comparable to the response observed after injection of endogenous mouse myoblasts genetically engineered to secrete high levels of the potent angiogenic factor vascular endothelial growth factor-A (VEGF-A). However, the quality and density of blood vessels induced by PEG was better than that observed by injected cells which expressed VEGF-A from transfected vectors.

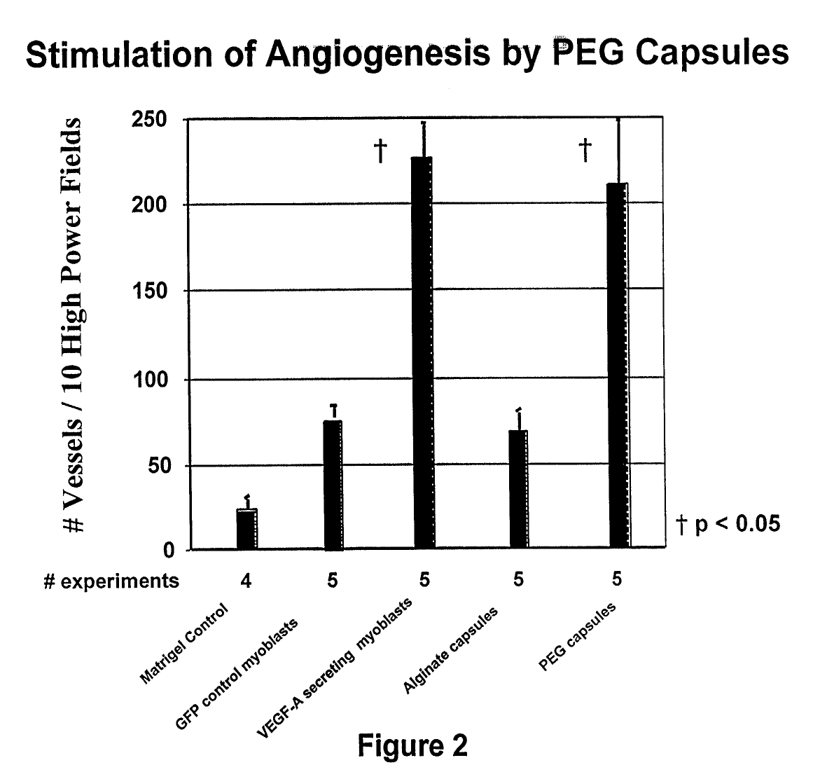

### SUMMARY OF THE INVENTION

The present invention provides novel methods and compositions for promoting tissue angiogenesis using PEG polymers either alone or combined with therapeutic angiogenic proteins or other therapeutic (e.g., gene therapy) approaches. According to the methods of the invention, PEG polymers are contacted directly with selected tissue areas (e.g., by injection) in an amount effective to induce or enhance angiogenesis within the area. Angiogenesis is promoted either directly by application of the PEG polymer and any accompanying angiogenic proteins contained in or applied with the PEG polymer, or indirectly by PEG-induced recruitment of monocytes to the tissue area which then secrete angiogenic proteins which promote angiogenesis.

Typically, PEG polymers for use in the invention are formed around a molecular matrix. In one embodiment, the matrix is made up in part of alginate/poly-L-lysine/alginate or, alternatively, agarose/poly-L-lysine/alginate. The matrix can be freely associated with the PEG or can be in the form of a core, optionally encapsulating angiogenic proteins or genes encoding such proteins, surrounded by the PEG. In another embodiment, the matrix is made up of heparin sepharose beads which optionally contain angiogenic proteins or genes encoding such proteins.

Suitable angiogenic proteins to be applied in conjunction with the PEG polymers include, for example, M-CSF, GM-CSF, VEGF-A, VEGF-B, VEGF-C, VEGF-D, basic FGF, PDGF-B, Angiopoietin 1, Angiopoietin 2, erythropoietin, BMP-2, BMP-4, BMP-7, TGF-beta, IGF-1, Osteopontin, Pleiotropin, Activin, and Endothelin-1. These proteins, or genes encoding these proteins, can be applied directly with the PEG polymers, or applied by sustained release from the PEG polymers (i.e., be incorporated into the PEG polymers). Suitable expression vectors for gene therapy application include, for example, adenoviral vectors, retroviral vectors, lentiviral vectors, RNA vectors, DNA vectors, naked DNA, liposomes, cationic lipids, AAV, and transposons.

Methods and PEG polymer compositions of the present invention can be used to promote angiogenesis in a safe and controlled manner in a variety of selected localized tissue areas. Accordingly, such methods and compositions can be used to treat a variety of tissue ischemias, including myocardial ischemia, ischaemic bowel disease, and peripheral ischemia.

### DETAILED DESCRIPTION OF THE INVENTION

The depletion of oxygen supply to due to obstructed or inadequate blood supply is the common pathological state associated with tissue ischemia, including myocardial ischemia, ischaemic bowel disease, and peripheral ischemia. The alleviation of the ischemic condition, and its attendant pathologies such as hypoxia, is critically dependant upon the process of angiogenesis, whereby new capillaries are generated from existing vasculature.

The present invention provides novel methods and compositions for achieving this goal using PEG polymers, optionally in conjunction with other angiogenic agents, to promote or enhance angiogenesis at selected localized tissue areas. Accordingly, the methods and compositions of the invention can be used to treat a variety of tissue ischemias and related conditions.

In one embodiment, the present invention provides a method of promoting angiogenesis at a selected tissue area by contacting the areas with a PEG polymer made up of at least two polymerized monomers, formed using a combination of the following reagents:

1) a photoinitiator,

2) a polymerizable PEG compound,

3) optionally at least one co-catalyst, and

4) optionally at least one reaction accelerator.

These components are mixed in varying combinations and then exposed to photo-radiation to activate the photoinitiators and, thus, initiate polymerization. A network then forms as the monomers polymerize into a three-dimensional PEG polymer.

As used herein, the term “polymerizable monomer” includes a molecular moiety which has one or more groups which allow, under certain condition, a covalent bond to form between a group on one monomer, and corresponding group on another monomer. Suitable monomers which can be used in the present invention include the family of polyethylene glycol (PEG) compounds, referred to herein interchangeably as PEG, PEG polymers and PEG compounds. PEG compounds are polymeric molecules comprising a variable-length backbone formed of multiple linked ethylene groups. As such, PEG compounds are available in a range of molecular weights, depending on the number of ethylene groups in the backbone.

In a preferred embodiment, PEG compounds which contain one or more acrylate groups serve as polymerizable monomers. Accordingly, polyethylene glycol monoacrylate, polyethylene glycol diacrylate, polyethylene glycol triacrylate, and polyethylene glycol tetraacrylate are preferred polymerizable monomers of the invention.

As used herein, the term “photoinitiator” includes molecules which are activated when exposed to certain wavelengths of photo-energy and can catalyze certain reactions when in an activated (excited) state. Suitable and preferred photoinitiators of the present invention include, for example, Eosin dyes and, particularly, Eosin Y (CAS number 15086-94-9).

As used herein, the term “cocatalyst” includes molecules which aid in the polymerization of monomers into PEG polymer. Suitable and preferred cocatalysts of the present invention include triethanolamine (TEOA). As further used herein, the term “reaction accelerator” includes molecules whose presence accelerates the polymerization of monomers into PEG polymer. Suitable and preferred reaction accelerators of the present invention include n-vinyl pyrrolidine.

Methods and techniques for producing the above-described PEG polymers which can be used in the present invention are described in U.S. Pat. No. 5,801,033, incorporated by reference in its entirety herein.

PEG polymers used in the invention can be formed in any dimentional manner around a matrix. The term “matrix” as used herein, refers to a molecular structure which serves as a scaffold upon which the PEG polymers are formed. They also generally contain reagents necessary for polymerization, as well as therapeutic compounds, if desired. In one embodiment, the matrix functions as a capsule which is surrounded by the PEG. In another embodiment, the PEG is intertwined with the matrix. As used herein, a “capsule” refers to a core around which polymerized PEG forms. Optimally, the matrix is comprised of material which is compatible (e.g., integratable) with PEG polymers of the invention, e.g., has a molecular structure that is amenable to PEG polymerization upon and/or throughout its volume, e.g., a matrix molecule. Suitable and preferred materials for use as the matrix (e.g., the capsule) include, for example, alginate, alginate/poly-L-lysine/alginate, and agarose/poly-L-lysine/alginate.

In another embodiment, the matrix comprises a “bead”, such as a heparin sepharose bead, which contains (e.g., has absorbed) the necessary reagents for polymerization, in addition to therapeutic agents, if desired. Accordingly, the bead can serve the dual purpose of acting as a scaffold and as a vehicle to deliver therapeutic molecules or compounds, e.g., angiogenic compounds, to selected tissue areas. Ideally, the bead is composed of inert or biocompatible material and has dimensions that are appropriate for injection into tissues. The bead also may be coated with a material to render it suitable as a delivery vehicle for a particular therapeutic compound. In a particular embodiment, the bead itself is coated with another matrix molecule (e.g., alginate/poly-L-lysine/alginate and/or agarose/poly-L-lysine/alginate) which is, in turn, polymerized with PEG. Suitable and preferred beads for use in the invention include, for example, agarose, sepharose, or cellulose beads. In a preferred embodiment, the beads are heparin and/or heparin-sepharose beads.

PEG polymers of the invention can be administered in conjunction with angiogenic factors to induce angiogenesis at selected tissue areas. This can be achieved by coadministering the angiogenic factor separately (either simultaneously or sequentially) with the PEG polymer, or by incorporating the angiogenic factor into the PEG polymer. For example, the angiogenic factor, or a gene encoding the factor, can be absorbed or encapsulated by the polymer matrix to provide controlled, sustained release of the factor from the PEG polymer.

As used herein, the term “angiogenic factors” includes proteins, factors, peptides and small molecule compounds which are able to induce or enhance angiogenesis. Suitable and preferred angiogenic proteins for use in the invention include, for example, proteins that are known in the art including M-CSF, GM-CSF, VEGF-A, VEGF-B, VEGF-C, VEGF-D, basic FGF, PDGF-B, Angiopoietin 1, Angiopoietin 2, erythropoietin, BMP-2, BMP-4, BMP-7, TGF-beta, IGF-1, Osteopontin, Pleiotropin, Activin, Endothelin-1 and combinations thereof.

Angiogenic factors can also be delivered in the form of genes encoding the factors. Expression vectors which contain one or more genes which encode complete or partial angiogenic factors can be combined with (e.g., incorporated into or delivered concurrently with) PEG polymers of the invention, as described above. Suitable expression vectors for transferring functional genetic elements (e.g. genes for angiogenic factors) into tissue and/or cells in accordance with the embodiments described herein are well known in the art and include, for example, adenoviral vectors, retroviral vectors, RNA vectors, DNA vectors, naked DNA vectors, lentiviral vectors, adeno-associated virus (AAV) and transposons (see, for example, Chapter 9 of Ausubel et al, Current Protocols in Molecular Biology, John Wiley & Sons, N.Y. (1989)). Methods for introducing these vectors into tissue and/or cells are also well known in the art. For example, transfection techniques which utilize liposomes, cationic lipids, DEAE dextran, and calcium phosphate/nucleic acid precipitates (see, for example, Chapter 9 of Ausubel et al Current Protocols in Molecular Biology, John Wiley & Sons, N.Y. (1989)).

Angiogenesis-promoting PEG polymer compositions of the invention can be delivered to localized tissue areas using a variety of art-recognized techniques, such as injection, implantation or mechanical delivery using, for example, a suitable catheter or stent. Accordingly, methods of the invention can be used to treat a variety of tissue ischemias, including, for example, myocardial and peripheral tissue ischemia.

This invention is further illustrated by the following examples which should not be construed as limiting. The contents of all references, patents and published patent applications cited throughout this application are incorporated herein by reference.

### EXAMPLES

### Example 1

PEG Polymers Can Induce or Enhance

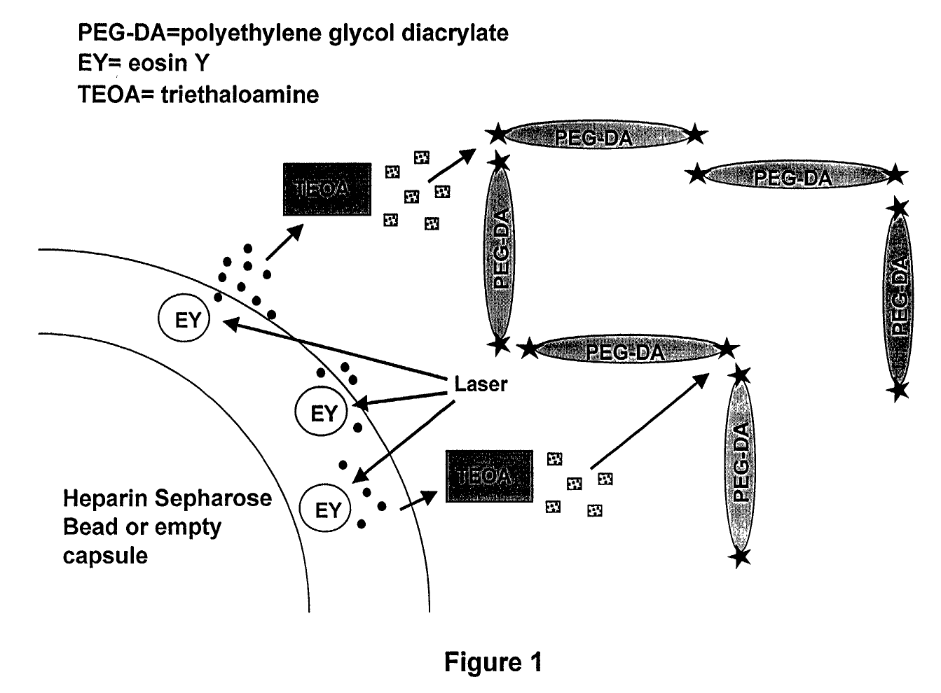

A variety of different polymers, one of which is shown in FIG. 1, were tested for their ability to encapsulate non-autologous cells such as monocytes or other cell-types to protect them from the immune system, thus aiding their use in treating ischemia. It was observed, unexpectedly, that one of the polymer compositions, PEG, was able to potently stimulate angiogenesis by itself when injected into animal models of ischemia. Moreover, the quality and density of the newly developed vessels was superior than those stimulated through injection of purified angiogenic proteins themselves, or vectors encoding the proteins. In particular, the quality and density of blood vessels induced by PEG was superior to that observed by injected cells which expressed VEGF-A from transfected vectors.

Equivalents

Those skilled in the art will recognize, or be able to ascertain using no more than routine experimentation, many equivalents of the specific embodiments of the invention described herein. Such equivalents are intended to be encompassed by the following claims.

What is claimed is:

1. A method of promoting angiogenesis comprising contacting a localized area of tissue with a PEG polymer in an amount effective to induce angiogenesis within the area of tissue.

2. The method of claim 1, wherein the PEG polymer further comprises a matrix.

3. The method of claim 2, wherein the matrix is selected from the group consisting of alginate, alginate/poly-L-lysine/alginate, and agarose/poly-L-lysine/alginate.

4. The method of claim 2, wherein the matrix comprises heparin sepharose beads.

5. The method of claim 3, wherein the matrix is in the form of a capsule which is surrounded by the PEG polymer.

6. The method of claim 1, further comprising contacting an angiogenic protein, or an expression vector encoding an angiogenic protein, with the area of tissue.

7. The method of claim 4, wherein the heparin sepharose bead contains an angiogenic protein or an expression vector encoding an angiogenic protein.

8. The method of claim 5, wherein the matrix core contains an angiogenic protein or an expression vector encoding an angiogenic protein.

9. The method of any one of claims 6, 7 or 8, wherein the angiogenic protein is selected from the group consisting of M-CSF, GM-CSF, VEGF-A, VEGF-B, VEGF-C, VEGF-D, basic FGF, PDGF-B, Angiopoietin 1, Angiopoietin 2, erythropoietin, BMP-2, BMP-4, BMP-7, TGF-beta, IGF-1, Osteopontin, Pleiotropin, Activin, and Endothelin-1.

10. The method of any one of claims 6, 7 or 8, wherein the expression vector is selected from the group consisting of adenoviral vectors, retroviral vectors, RNA vectors, DNA vectors, naked DNA, liposomes, cationic lipids, lentiviral vectors, AAV, and transposons.

11. The method of claim 1, wherein the PEG polymer is contacted with the tissue area by injection.
